(19) United States
(12) Patent Application Publication
(10) Pub. No.: US 2025/0342955 A1
(43) Pub. Date: Nov. 6, 2025

(54) SYSTEM AND METHOD FOR CALL LIGHT MONITORING

(71) Applicants: Michael Forsgren, Yorba Linda, CA (US); Robert Horn, Yorba Linda, CA (US)

(72) Inventors: Michael Forsgren, Yorba Linda, CA (US); Robert Horn, Yorba Linda, CA (US)

(21) Appl. No.: 18/653,916

(22) Filed: May 2, 2024

(51) Int. Cl. G16H 40/67

(52) U.S. Cl. CPC G16H 40/67

(57) ABSTRACT

The present disclosure relates to a call light monitoring system designed to enhance response efficiency and patient care management in healthcare facilities. This system includes unobtrusive monitoring capabilities that detect the activation of call lights without interfering with existing facility infrastructure. The system features a real-time display that provides immediate visual feedback on the duration of active call lights, enabling staff to prioritize responses based on urgency and elapsed time. Additionally, the system includes a comprehensive reporting module that records and analyzes the history of call light durations. This historical data can be used for performance evaluations, optimizing staff allocations, and identifying patterns that may indicate areas needing process improvement. The integration of these features into a single system provides a robust tool for improving patient satisfaction and operational efficiency in healthcare settings.

### FIELD OF THE INVENTION

The current invention is a healthcare call light monitoring system.

### BACKGROUND

Healthcare facility call light systems are critical for patient care as they provide a direct method for patients to alert nursing staff when assistance is needed. Traditionally, these systems consist of a light panel that indicates which rooms have activated a call light. While functional in basic operation, these systems possess significant limitations that impact response efficiency and patient satisfaction.

The majority of existing call light systems found in healthcare facilities are outdated, some having been in place for many decades. These antiquated systems typically only indicate the presence of an active call without providing any information on the duration of the wait. As a result, nursing staff must often respond to calls without knowledge of which requests are most urgent, making it challenging to prioritize patients effectively. This can lead to inefficiencies in response times and variations in patient care quality.

While a small percentage of these systems have been retrofitted with monitoring capabilities, such enhancements generally remain inadequate. These retrofitted systems are often based on wired technologies and offer only post-hoc reports detailing response times for individual rooms and facility-wide averages. Notably, such systems lack a real-time display of active call light durations, which are crucial for immediate assessment and prioritization of patient needs. Additionally, the reliance on average call times for performance metrics can be misleading. These averages may obscure underlying issues such as consistently delayed responses to certain patients or rooms, thus failing to provide actionable insights for improving service quality and operational efficiency.

Furthermore, there is a significant advantage to implementing a non-intrusive monitoring system, as it offers universal applicability and compatibility with a diverse array of existing call light systems, regardless of their age or design. A non-intrusive approach does not require extensive modifications to the current infrastructure, which not only reduces installation costs significantly but also accelerates the deployment process. This is particularly beneficial for healthcare facilities that are constrained by limited budgets and cannot afford extensive downtime or disruptions that typically accompany comprehensive system overhauls. By overlaying a modern monitoring solution onto the existing setup, facilities can upgrade their service capabilities without the high costs or logistical challenges associated with replacing old systems. This makes the adoption of advanced monitoring technologies more feasible and attractive, facilitating widespread improvements in patient care efficiency across the healthcare industry.

The current state of call light systems indicates a clear need for an innovative solution that not only modernizes the existing infrastructure but also enhances the functionality to meet the demands of contemporary healthcare environments. Such improvements should aim to affordably provide real-time data on call light durations, enabling a more strategic and patient-centric response approach.

### SUMMARY

The present invention relates to a sophisticated monitoring system designed to enhance the operational efficiency and response effectiveness in healthcare facilities or environments requiring call light management. The system comprises a series of non-intrusively deployed monitoring devices, each equipped to detect the state of one or more call lights without requiring direct electrical connections to existing call light circuitry. Utilizing cameras or other light-sensing technologies, these monitoring devices are capable of discerning multiple call light states and calculating the duration each light remains in a given state.

Key to the functionality of the system is its ability to generate and communicate real-time and historical data regarding call light states via a designated communication path. This data is then displayed on one or more display devices, providing actionable insights into patient needs and staff responses. The system is further enhanced by features that allow monitoring devices to asynchronously transmit both current state and historical duration data over communication paths to remote databases or display devices. This allows for centralized or remote monitoring and management of call light activity, ensuring that urgent patient needs are met promptly and efficiently.

Additionally, the system includes provisions for implementing advanced task management. Monitoring devices can interact with a database of periodically required tasks to alert staff of pending tasks through various interfaces, such as kiosks or web browsers. These devices can also initiate SMS text messages under specific conditions related to the duration, time, and associated individual or location of a call light, ensuring critical alerts are communicated swiftly.

Overall, this system provides a comprehensive solution for managing call light systems and associated tasks, significantly improving response times, staff coordination, and overall patient care in healthcare settings.

### BRIEF DESCRIPTION OF THE DRAWINGS

FIG. 1A illustrates an example of a call light monitoring device positioned above a health care facility's wall-mounted call light panel.

FIG. 1B illustrates a side view of FIG. 1A

FIG. 2 illustrates a call light monitoring system for a health care facility that has one call light panel, a wireless network with internet connectivity, and two nursing stations that each require a display.

FIG. 3 illustrates a call light monitoring system for a health care facility where two call light panels must be monitored in order to view the call lights for all the rooms. Additionally, a kiosk device is shown displaying tasks to be completed.

### DETAILED DESCRIPTION

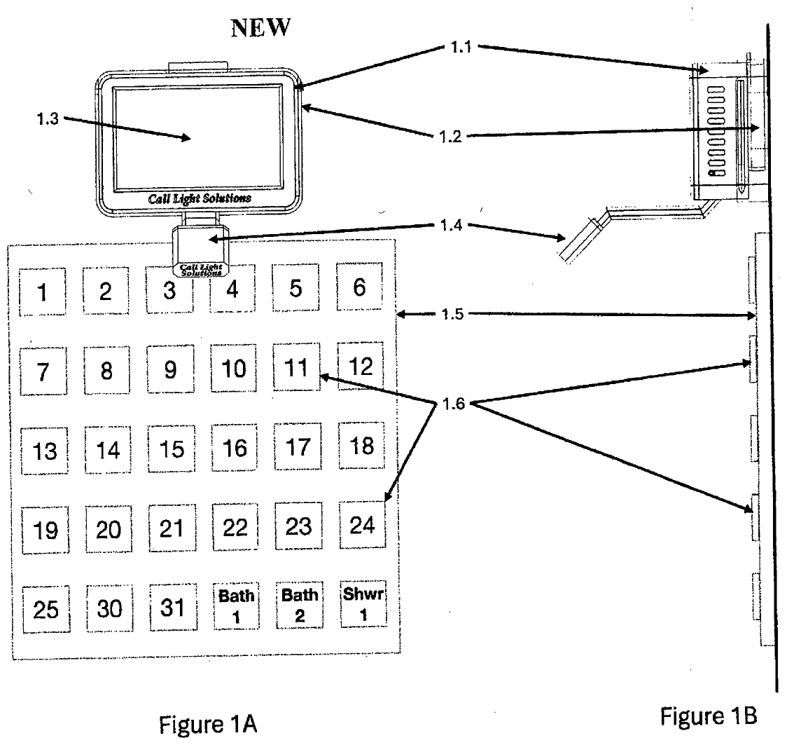

FIGS. 1A and 1B provide a front and side view, respectively, of one embodiment of a call light monitoring device 1.1 and a typical health care facility call light panel 1.5. The monitoring device 1.1 is placed in a mounting bracket 1.2 that is attached to a wall above a health care facility call light panel 1.5.

The monitoring device 1.1 includes a display screen 1.3 that can be used to provide status information such as internet connectivity as well as installation instructions and real-time video helpful in determining the physical placement of the device. During normal operation, the display screen 1.3 indicates status of the call light monitoring system status. The monitoring device 1.1 also has a sensor mounting arm 1.4 containing a camera or other light sensing device configured to determine the state of each individual call light (e.g. 1.6) on the call light panel 1.5.

Typical call light 1.6 states in most facilities include solid off, solid on, and blinking; where blinking indicates the call was initiated from the bathroom associated with the room number. The monitoring device 1.1 distinguishes between these states to properly create both active and historical call light data. An entry in active call light data consists of call light designation (e.g. room number), active state (e.g. solid on or blinking), and a timestamp of when that state was entered. When the state of an active call light changes to a different state, the monitoring device deletes the corresponding active call light entry and creates a new entry in the historical call light data which includes call light designation, active state, timestamp of when the active state was exited, and the number of seconds the call light had been in the active state.

Additionally, if the active state changes from one active state to another active state (e.g. solid on to blinking or blinking to solid on), the monitoring device also creates a new entry in the active call light data for the corresponding call light. The monitoring device maintains both the active and historical call light data in a data base. The database is configured to maintain its contents even if power is interrupted. In one embodiment this database could be a mySQL server running on the monitoring device 1.1.

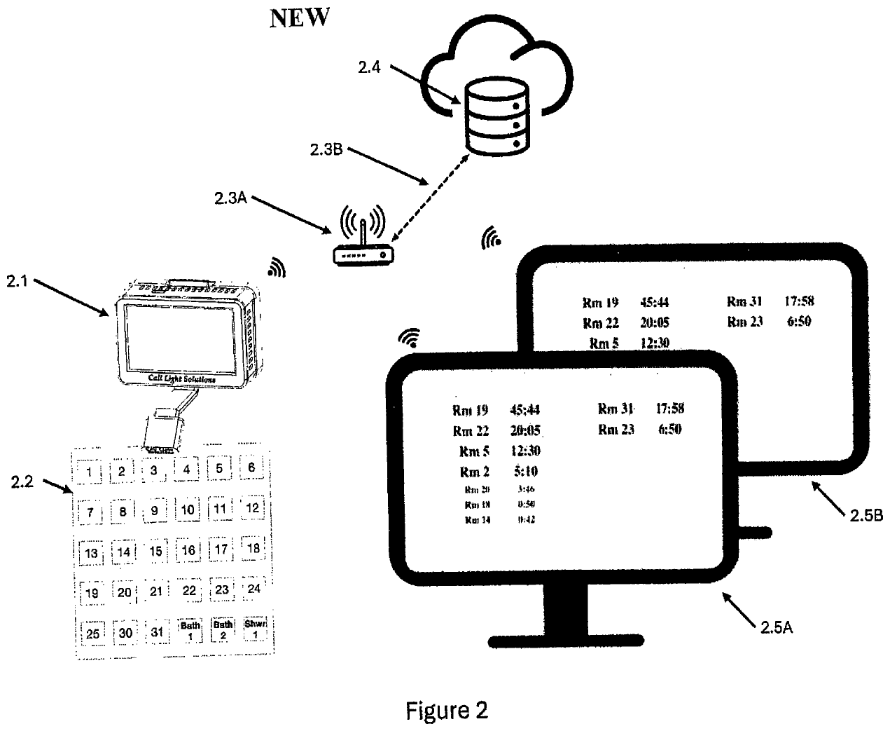

FIG. 2 illustrates one example of a call light monitoring system installed in a typical health care facility. In this example, 2.1 is the call light monitoring device described in FIGS. 1 and 2.2 represents the facility's call light panel. Further illustrated is a WiFi connection between the call light monitoring device 2.1 and a facility wireless router 2.3A that provides communication path for local WiFi-enabled devices to communicate with each other. A wired connection to the internet 2.3B additionally allows local WiFi devices to communicate with Internet addresses, including a cloud database 2.4. The system also includes two Wi-Fi connected display units (2.5A and 2.5B) which could be placed in the facility near nursing stations (e.g. North and South).

In the FIG. 2 system, the monitoring device 2.1 is configured to provide a list of active call lights and current active durations to the display units 2.5A/B via the WiFi communication channel. In one embodiment, the monitoring device 2.1 implements a local web server and the display units 2.5A/B each run a web browser. The web page is periodically updated such that the duration time and list of active call lights matches the state of the call light panel as it changes periodically. The system supports the ability to display all the call light data for the entire facility on each monitor unit (2.5A/B) or optionally display only active call lights for the rooms assigned to specific stations (e.g. North or South). The FIG. 2 example also illustrates the ability for the call monitoring device 2.1 to segregate by columns the display of call light data according to nurse station assignment and order the call light data such that longer durations are listed above lower duration active calls. Call monitoring devices 2.1 can also be configured to use text color, background highlight color, and text size to further differentiate call lights by their urgency relative to active duration.

One attribute of this system is that full display functionality is maintained even if internet connectivity 2.3B is lost or the ability to connect to the cloud database 2.4. However, if internet connectivity to the cloud database is available, the call monitoring device is configured to synchronize the local database of both active call data and historical call data to the cloud database 2.4. If the connectivity is interrupted, the call light monitoring device will resynchronize the cloud database 2.4 to the local call light data. This behavior enables cloud applications to display active call lights and also generate various reports based on historical call light data.

When Internet connectivity exists, the call light monitoring device 2.1 can be configured to send SMS text messages to specific destinations based on criteria that may include times of day, room number(s), and call light active duration(s). Internet connectivity also allows the call light monitoring device 2.1 to implement cloud-based configuration requests to modify settings which may include texting parameters or the manner in which active and historical call light data is shown on the display units 2.5A/B.

Furthermore, Internet connectivity allows the call light monitoring device 2.1 to be controlled remotely. This feature enables remote initial system setup, software updates, remote tuning, and remote debugging. These capabilities are used to maintain, optimize, and improve the call light monitoring system and extends its usable lifetime.

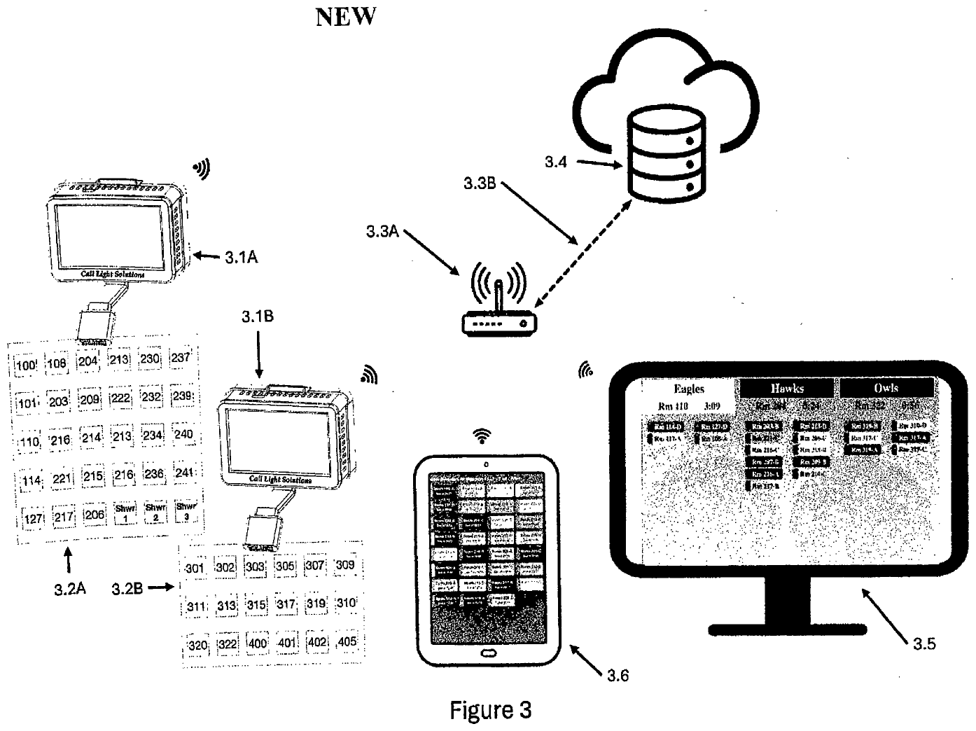

FIG. 3 illustrates a call light monitoring system for a facility where two call light panels 3.2A/B are observed by two call light monitoring devices 3.1A/B because neither call light panel, 3.2A nor 3.2B, includes all the call lights required for the entire facility. Note that in this example, room numbers indicated on call light panel 3.2A are not repeated on 3.2B and vise versa; however, some facilities exist where a subset of call light room numbers are duplicated between multiple call light panels. In FIG. 3, call light monitor 3.1A is configured as the master and 3.1B is configured as a slave.

The role of the slave call light monitor 3.1B is to generate both active and historical call light data for the call lights on its associated panel 3.2B and then mirror that data to the master call light monitor 3.1A via local WiFi communication 3.3A and also to the cloud database 3.4 via internet connectivity 3.3B. Additionally, the slave call light monitor may support SMS text message generation for call lights on its associated call light panel 3.2B as well as remote control and remote configuration capabilities previously described in paragraphs [0021] and [0022]. In general, when a facility has N unique (i.e. non-identical room numbers) call light panels, the call light monitoring system will have one master and N-1 slave call light monitoring devices.

When one or more slave call light monitors exist, the master call light monitoring device 3.1A is configured to aggregate data from its associated call light panel 3.2A with all active and historical call light data received from slave call light monitoring devices (e.g. 3.1B). The master 3.1A is also configured to be the one and only provider of call light display data to any display units (e.g. 3.5). In one embodiment, the master 3.1A also limits SMS message generation to those rooms shown on its associated call light panel 3.2A.

FIG. 3 further illustrates an optional capability to integrate display and management of periodic tasks with call light monitoring and management. In one embodiment, call light monitoring device 3.1A, configured as master or standalone, synchronizes with a cloud database 3.4, a list of periodic health care tasks. Examples of tasks may include but are not limited to taking a measurement of a patient's blood pressure, assisting in bladder training, moving a patient to an alternate position in bed to avoid bed sores, providing a nutritional supplement between meals, or assisting a patient to bathe. Example configurations of period of repetition could be daily, hourly, certain days of the week, or specific times each day.

In FIG. 3, the display unit 3.5 illustrates, in addition to active call lights and durations, a series of task reminders segregated by assigned nurse station (e.g. Eagles, Hawks, Owls) and ordered by urgency level. In this embodiment, a reminder appears on the display unit 3.5 only when the corresponding task is ready to be performed and is removed from view once the task has been completed. Each reminder has a primary background color that corresponds to the type of task to be performed and a tag color on the left of the reminder symbol that is color coded based on urgency.

The call light monitoring system is further configured to include at least one kiosk device 3.6. In this embodiment the kiosk 3.6 is a tablet computer configured to both display unresolved tasks and accept input recording task resolution of the task. Data required to resolve the task may include how the task was resolved (e.g. completed, patient refused, patient not available) and the identity of the caregiver that resolved the task (e.g. nurse initials). The time of the resolution may be inferred to be when the resolution was input into the kiosk. Alternate interfaces may also be used to record task resolution such as cell phone or computer terminal.

What is claimed is:

1. A system, comprising:
at least one monitoring device configured to detect the state of at least one call light without being electrically connected to the call light circuitry;
a communication path configured to transmit information generated by the monitoring device;
at least one display device capable of reporting information generated by the monitoring device.

2. The system in claim 1, wherein the monitoring device uses a camera or other light sensing device to detect the state of at least one call light.

3. The system in claim 1, wherein the monitoring device distinguishes two or more call light states.

4. The system in claim 1, wherein the monitoring device calculates the duration of time that at least one call light is consistently in at least one state.

5. The system in claim 1, wherein the monitoring device maintains both current call light state duration and history data of call light state durations for at least one call light.

6. The system in claim 1, wherein the monitoring device is configured to respond to a request to transmit current state duration and history data for at least one call light over a communication path.

7. The system in claim 1, wherein the monitoring device is configured to asynchronously transmit current state duration and history data for at least one call light over a communication path to a remote database or remote display device.

8. The system in claim 1, wherein one or more monitoring devices are configured as slave devices that asynchronously transmit current state duration and history data for at least one call light each to a designated master monitoring device that aggregates and transmits the current state and history data from all monitoring devices in the system.

9. The system in claim 1, wherein the monitoring device provides a web browser interface for transmitting call light current state and history data.

10. The system in claim 1, where the monitoring device is configured to initiate one or more SMS text messages based on one or more of the following preselected conditions:
The duration of the call light state exceeds a specified threshold.
The time of day.
The day of the week.
The location or individual associated with the call light.

11. The system in claim 1, wherein the monitoring device is configured to scan a database of periodically required tasks associated with one or more individuals and transmit a list of tasks that are currently due and not yet resolved.

12. The system in claim 1, wherein a kiosk device is configured to display tasks that are currently pending and not yet resolved and accept resolution parameters including one or more of the following:
The time of resolution of a task.
The type of resolution of a task. Allowed types may include task completed, task refused by individual, individual not available.
The identity of the worker who resolved the task.

13. A monitoring device in claim 11 that receives and stores task-specific user input data from a kiosk device and uses that information to determine which tasks are currently pending and not yet resolved.

14. A call light monitoring method, comprising:
detecting the state of at least one call light without being electrically connected to the call light circuitry;
transmitting detected call light information;
reporting information generated by the monitoring device.

15. The method in claim 14, further comprising: using a camera or other light sensing device to detect the state of at least one call light.

16. The method in claim 14, further comprising: distinguishing two or more call light states.

17. The method in claim 14, further comprising: calculating the duration of time that at least one call light is consistently in at least one state.

18. The method in claim 14, further comprising: maintaining both current call light state duration and history data of call light state durations for at least one call light.

19. The method in claim 14, further comprising: responding to a request to transmit current state duration and history data for at least one call light over a communication path.

20. The method in claim 14, further comprising: asynchronously transmitting current state duration and history data for at least one call light over a communication path to a remote database or remote display device.

21. The method in claim 14, further comprising: configuring slave devices that asynchronously transmit current state duration and history data for at least one call light each to a designated master monitoring device that aggregates and transmits the current state and history data from all monitoring devices in the system.

22. The method in claim 14, further comprising: providing a web browser interface for transmitting call light current state and history data.

23. The method in claim 14, further comprising: initiating one or more SMS text messages based on one or more of the following preselected conditions:
The duration of the call light state exceeds a specified threshold.
The time of day.
The day of the week.
The location or individual associated with the call light.

24. The method in claim 14, further comprising: scanning a database of periodically required tasks associated with one or more individuals and transmitting a list of tasks that are currently due and not yet resolved.

25. The method in claim 14, further comprising: displaying tasks that are currently pending and not yet resolved and accepting resolution parameters including one or more of the following:
The time of resolution of a task.
The type of resolution of a task. Allowed types may include task completed, task refused by individual, individual not available.
The identity of the worker who resolved the task.

26. The method in claim 14, further comprising: receiving and storing task-specific user input data from a kiosk device and uses that information to determine which tasks are currently pending and not yet resolved.
